(19) United States
(12) Patent Application Publication
(10) Pub. No.: US 2011/0082091 A1
(43) Pub. Date: Apr. 7, 2011

(54) Method for Preclinical Testing of Immunomodulatory Drugs

(75) Inventor: Thomas Hünig, Winterhausen (DE)

(73) Assignee: TheraMAB GmbH, Wurzburg (DE)

(21) Appl. No.: 12/892,604

(22) Filed: Sep. 28, 2010

(30) Foreign Application Priority Data
Sep. 28, 2009 (EP) EP 09 01 2276.3
May 25, 2010 (EP) EP 10 00 5421.2

(51) Int. Cl. A61K 38/13; C07D 498/16; C07J 9/00; C07K 7/64; A61K 31/56; A61P 37/06; C12Q 1/02; G01N 33/53

(52) U.S. Cl. 514/20.5; 540/456; 540/120; 530/321; 514/171; 435/29; 435/7.1

(57) ABSTRACT

The invention teaches a method for testing a prospective or known immunomodulatory drug for T-cell activation, comprising the step of contacting in-vitro a peripheral blood mononuclear cell (PBMC) culture with a predetermined amount of the prospective or known immunomodulatory drug and observing the PBMC culture for T-cell activation using a readout system, upon contact with the prospective or known immunomodulatory drug, wherein the cell density of a PBMC preculture is adjusted such that cell-cell contact of the PBMC is enabled and wherein the PBMC preculture is cultured for at least 12 h.

This application claims the benefit of EP 10 00 5421.2, filed May 25, 2010 and EP 09 01 2276.3, filed Sep. 28, 2009, which are hereby incorporated by reference in their entirety.

The invention relates to a method for testing a prospective or known immunomodulatory drug for T-cell activation, comprising the step of contacting in-vitro a peripheral blood mononuclear cell (PBMC) culture with a predetermined amount of the prospective or known immunomodulatory drug and observing the PBMC culture for T-cell activation using a readout system, upon contact with the prospective or known immunomodulatory drug, wherein the cell density of a PBMC preculture is adjusted such that cell-cell contact of the PBMC is enabled and wherein the PBMC preculture is cultured for at least 12 h. The invention further relates to a method of testing cytokine storm attenuating drugs in-vitro.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Immunotherapeutic drugs which modulate the activity of lymphocytes are preclinically evaluated in two systems: Animal models, usually rodents, and, if available, primate; and cultures of human peripheral blood mononuclear cells (PBMC).

PBMCs are routinely used because, first, they contain all subsets of lymphocytes as well as monocytes, and, second, they are readily available from venous blood drawn from healthy donors or from patients. In vitro stimulation of these PBMCs is considered a useful indicator of the activities of an immunomodulatory drug to be expected in vivo.

Certain T-cell activating agents, in particular monoclonal antibodies (mAb) addressing the T-cell antigen receptor (TCR) such as OKT3, the first mAb used in the clinic for immunosuppression, induce the systemic release of proinflammatory cytokines (Abramowicz et al., 1989). The most dangerous of these are TNF, interferon-gamma (IFN-gamma) and IL-2. In patients receiving mAb therapies, control of such a cytokine release syndrome or “cytokine storm” is routinely achieved by high dose corticosteroid treatment.

TGN1412 is a humanized monoclonal antibody (mAb) of the IgG4 subclass specific for the costimulatory molecule CD28 expressed by human T-cells. It is called a “CD28 superagonist” (CD28SA) because unlike the classic CD28-specific mAb, it can activate T-lymphocytes without simultaneous engagement of the T-cell antigen receptor (TCR) (Hunig, 2007). TGN1412 was developed by the now defunct TeGenero AG, Würzburg.

During a first-in-man trial conducted by the independent Parexel Clinical Trial Unit at Northwick Park Hospital, London, on Mar. 13, 2006, intravenous application of 100 μ/kg body weight of TGN1412 to healthy human volunteers led to a life-threatening cytokine release syndrome that was only controlled after transfer of the volunteers to the hospital's intensive care unit (Suntharalingam et al., 2006).

The preclinical work presented by the sponsor, TeGenero AG, showed no evidence for such a “cytokine storm” in an analogous rat model using a rat-CD28-specific superagonist, and in cynomolgus monkeys receiving TGN1412 itself at up to 50 fold higher doses than the human volunteers (Duff, 2006). Furthermore, addition of TGN1412 to cultures of human PBMCs also did not result in cytokine release. All key monkey and PBMC culture experiments were repeated by the British National Institute for Biological Standards and Control (NIBSC) acting on behalf of the government's Expert Scientific Group on Phase One Clinical Trials, and confirmed the innocuous behaviour of TGN1412 in these systems (Duff, 2006). Three years after the failed TNG1412 trial, it still has not been clarified why this in vitro assay did not warn against the cytokine storm experienced by the human volunteers.

The failure of rodents and cynomolgous monkeys to release toxic systemic cytokines after injection of CD28SA is due to interspecies differences in the reactivity of the intact immune system to such agents, and specific suggestions for such differences have been made. (Gogishvili et al., 2009; Nguyen et al., 2006; Schraven and Kalinke, 2008).

These findings indicate that because of species-specific reaction patterns, even primate animal models are not always safe predictors of human reactivity to novel drugs.

A human being has roughly 10¹² T-lymphocytes, and less than one percent of these are circulating in the blood at any given moment. Therefore, the failure of cultured PBMCs to respond to TGN1412 is either due to a functional defect in these cells as compared to those residing in lymphoid tissues (which obviously responded with cytokine release in the volunteers), or to the requirement of a cell type present in lymphoid organs but not in blood for TGN1412-mediated activation of T-lymphocytes.

Reproduction of the cytokine storm observed in the human volunteers of the London TGN1412 trial in cell culture is, therefore, urgently needed to understand its mechanism and to test its sensitivity to pharmacological suppression.

From a broader point of view, the failure of known human PBMC cultures to respond to soluble TGN1412 with cytokine release indicates that this system does not respond to all lymphocyte-activating agents in the same fashion as does the intact human immune system inside the body. Correction of this defect may not only allow a detailed analysis of the effects of human CD28 superagonists (SA) such as TGN1412, but may also reveal the reactivity of other, seemingly innocuous drugs during preclinical development.

One embodiment of the invention is, accordingly, to provide improved means for in-vitro testing of immunomodulatory drugs, in particular CD28SA, with respect to potential cytokine storms. A further embodiment of the invention is to provide means for testing drugs suitable for attenuating cytokine storms.

A method for testing a prospective or known immunomodulatory drug for T-cell activation is provided. The method includes contacting in-vitro a peripheral blood mononuclear cell (PBMC) culture with a predetermined amount of the prospective or known immunomodulatory drug and observing the PBMC culture for T-cell activation using a readout system, upon contact with the prospective or known immunomodulatory drug, wherein the cell density of a PBMC preculture is adjusted such that cell-cell contact of the PBMC is enabled and wherein the PBMC preculture is cultured for at least 12 h.

In a preferred embodiment of the invention, the readout system observes the PBMC culture for release of at least one cytokine from the PBMCs, observes cell proliferation or is another suitable readout system, like changes in gene expression, protein expression and/or posttranslational modifications.

The term “immunomodulatory drug” preferably means any therapeutic agent that is capable to activate or suppress the immune system, e.g., by activating or inhibiting lymphocyte functions, in particular T-cell functions like T-cell inhibition or activation. Specific examples of immunomodulatory drugs are provided herein below.

The term “T-cell activation” preferably specifies the mechanisms of activation of T-cells which may vary slightly between different types of T cells. The “two-signal model” in CD4+ T cells, however, is applicable for most types of T-cells. In more detail, activation of CD4+ T cells typically occurs through the engagement of both the T cell receptor and CD28 on the T cell surface by the major histocompatibility encoded antigen-presenting molecule and with its bound antigenic peptide and B7 family members on the surface of an antigen presenting cell (APC), respectively. Both cell-cell contacts are generally required for the production of an effective immune response. For example, in the absence of CD28 co-stimulation, T-cell receptor signaling alone may result in T-cell anergy. The further signaling pathways downstream from both CD28 and the T cell receptor involve many further proteins known to the skilled person. The activation of T-cells may be determined by cytokine release and/or cell proliferation, in particular proliferation of T-cells, as described herein below.

The term “peripheral blood mononuclear cell (PBMC)” preferably defines any blood cell having a round nucleus, for example, lymphocytes and monocytes. These blood cells recirculate between tissues and blood, and are a critical component in the immune system to fight infection and adapt to intruders. The lymphocyte population of PBMCs typically consists of T cells (CD4 and CD8 positive ˜75%), B cells and NK cells (˜25% combined). PBMCs may be obtained from whole blood samples by methods well known in the art. For example, PBMCs may be extracted from whole blood using a ficoll gradient. Ficoll is a hydrophilic polysaccharide that separates layers of blood on the basis of density, with monocytes and lymphocytes forming a layer between plasma and the Ficoll solution. Besides whole heparinized blood as starting material, buffy coats (the layer of white cells ontop of erythrocytes obtained by centrifugation of whole blood), and the contents of leukoreduction chambers obtained as a bye-product of platelet concentrates in blood banks can be used.

The term “precultured” means that the PBMC culture is cultured in absence of immunomodulatory drugs and prior to contact with such drugs to be tested. Methods of culturing PBMCs are well known in the art and also described herein below.

The term “observe” comprises the qualitative, half-quantitative and quantitative measurement of concentrations of the at least one cytokine or of (cell) proliferation, preferably T-cell proliferation, or other cellular responses, like changes in gene expression, protein expression and/or posttranslational modifications, with known methods of the art.

One aspect of the invention is based on the surprising finding that a PBMC culture, which is prepared by standard methods, but further precultured for a predetermined period of time prior to contact with the immunomodulatory drug suddenly shows sensitivity with respect to cytokine release triggered by contact with immunomodulatory drugs, which do not trigger cytokine release in the absence of the preculture process. Another aspect of the invention is further based on the finding that this preculture effect is promoted by cell-cell contacts of the PBMCs for a predetermined period of time. In other terms, the PBMC culture should not be freshly prepared when the immunomodulatory drug is added.

Therefore, an embodiment of the invention is useful to predict the reactivity of individuals to immunomodulatory drugs, like TGN1412, and as it will be explained later in further detail, the ability of immunosuppressant drugs such as corticosteroids to control undesired reactions. It is also useful to further understand the mode of action of CD28SA (i.e. CD28 specific superagonistic monoclonal antibody). As mentioned above, CD28 is expressed on the surface of T-cells and co-stimulation via CD28 and the TCR is normally required for T-cell activation in a physiologal situation whereas direct T cell activation is also enabled by superagonistic antibodies to CD28. Moreover, the invention is useful in screening prospective immunomodulatory drugs for their T-cell activating potential, including cytokine release. Since the PBMCs recovered after high-density preculturing most likely reflect the status of T-cell reactivity found in the lymphoid organs, they should, in combination with an activating agent, also be employed to test immunosuppressant drugs because circulating T-cells may be more easily suppressed due to their “inactive” status, resulting in misleading results on the efficacy of such drugs.

In a preferred embodiment of the invention, the preculture step is carried out by storing the PBMC culture for at least 24 h, preferably at least 36 h, more preferably at least 45 h, at 35° C. to 40° C., preferably at 36° C. to 38° C., in absence of immunomodulatory drugs, and prior to the contact with the immunomodulatory drug to be tested.

In another preferred embodiment of the invention the cell density of the PBMC culture during the preculture step is at least 2*10⁶/ml, preferably at least 5*10⁶/ml, more preferably at least 10⁷/ml. In terms of cell density at the surface of the tissue culture vessel, it should be at least 4*10⁵/cm², preferably at least 10⁶/cm², most preferably at least 2*10⁶/cm². The values provided apply directly to vessel consisting of flat wells. In round wells or conical wells the overall density will differ in that the cell density is high at the bottom of the well and low in upper parts of the well. Accordingly the above given cell densities measured in volume shall refer to partial volumes in vessels of any kind as well, i.e. the given densities shall be provided in a partial volume of e.g. at least 10 μl magnitude of the total preculture volume present. Also any other method of achieving cell densities of viable cells, as claimed, shall be encompassed in the invention. The minimum number of cells being in cell-cell-contact preferably is at least 50.000.

In a further preferred embodiment the immunomodulatory drug is an immunostimulating drug, like an antibody, preferably a monoclonal antibody. Specifically, the monoclonal antibody can be a human CD28 specific superagonistic monoclonal antibody. The immunomodulatory drug under test can, however, without being limited thereto, also be selected from the group consisting of lectins, like concanavalin A (ConA), or phytohemagglutinine (PHA), natural extracts comprising lectins, like echinacea extract (e.g. Echinacin, Madaus, Germany), or mistletoe extract (e.g. Lektinol, Madaus, Germany), superantigens, like Staphylococcal Enterotoxin B (SEB), Staphylococcal Enterotoxin A (SEA), Toxic Shock Syndrome Toxin-1 (TSST-1), staphylococcal pyogens, like Staphylococcal Pyogene Enterotoxin B (SPEB), superantigens produced by mycoplasma, like Mycoplasma arthriditis, or by black plague bacteria, like Yersinia pseudotuberculosis, superantigens produced by certain pathogenic viruses, like EBV or HIV-1.

In another preferred embodiment of the invention the cytokine observed is selected from the group consisting of TNF, IFN-gamma, IL-1-beta, IL-1, IL-2, IL-3, IL-4, IL-5, IL-6, IL-7, IL-8, IL-9, IL-10, IL12p70, IL-13, IL-14, IL-15, IL-16, IL-17, IL-21, IL-35, and LT, and combinations thereof.

Alternatively proliferation of the cell can be observed, wherein proliferation is given, if the number of cells increases within a time unit by a predetermined amount. This amount can be chosen by the artisan by usual consideration. The cells that proliferate in this assay are preferably PBMCs and more preferably T-cells.

In a further preferred embodiment of the method for testing a prospective or known immunomodulatory drug for T-cell activation, the PBMC culture comprises memory T cells.

Memory T cells are a subset of antigen-specific T cells that have previously encountered and responded to their cognate antigen. Thus, the term antigen-experienced T cell is alternatively applied in the art. In this regard it is furthermore preferred that the memory T cells are CD45RO+ T cells. As it has been found by the inventors the cytokines released by the PBMCs in the method of the invention are predominately released by memory T cells which are comprised in the PBMCs.

In a further preferred embodiment of particular relevance with respect to the second-mentioned object, the precultured PBMC culture is additionally contacted with a prospective drug for attenuating the release of at least one cytokine (immunosuppressant drug), at the same time as contacting of the PBMC culture with the known immunomodulatory, in particular stimulating, drug or subsequently after a predetermined period of time, or a predetermined period of time there before, wherein the cytokine release is further observed. The predetermined period of time can be in the range of 10 s to 12 h, preferably in the range of 10 s to 1 h. In case the PBMC culture is additionally contacted with a immunosuppressant drug, it has to be understood that the immunosupressive effect can be determined in comparison to the state where the PBMC culture is contacted with a prospective or known immunomodulatory drug alone. The immunosuppressant drug can also be contacted with the precultured PBMC culture alone for example to determine its suppressive effect on natural, latent T cell activation or unnatural, elevated T cell activation, e.g. caused by an immune disorder or environmental agents.

The prospective drug for attenuating the cytokine release is preferably selected from the group consisting of corticosteroids, including, but not limited to dexamethasone or methylprednisolone.

The prospective drug for attenuating the cytokine release may further be another immunosuppressive drug selected from the group consisting of Rapamycin or calcineurin inhibitors, like cyclosporine A, Voclosporin, or Tacrolimus.

In this preferred embodiment it becomes possible to test in-vitro, whether a particular drug intended for attenuation of a cytokine storm (immunosuppressant drug) is suitable for this purpose, in particular in cases where a cytokine storm occurs in vivo, despite administration of the immunomodulatory drug at a concentration that would prima facie be considered to prevent a cytokine storm. In particular, this variant of the invention allows creating a matching pair of immunomodulatory drug and attenuating drug and provides secure means for managing unexpected cytokine storms in in-vivo experiments, in particular clinical trials with humans.

In this regard the term “cytokine storm”, which is also known as “hypercytokinemia” in the art, preferably defines a systemic inflammatory response in a patient inter alia characterized by hypotension, pyrexia and/or rigors, and potentially resulting in death. A cytokine storm is presumably caused by an uncontrolled positive feedback loop between cytokines and immune cells, resulting in highly elevated levels of various cytokines.

In a further embodiment the invention relates to a corticosteroid, Rapamycin and/or a calcineurin inhibitor, for use in treating, attenuating or preventing a cytokine storm upon administration of an anti-human CD28 specific superagonistic monoclonal antibody.

In a preferred embodiment the antibody is TGN1412.

The amino acid sequence of the antibody TGN1412 has been described in WO-A2 2006/050949.

In a further preferred embodiment the corticosteroid is dexamethasone and/or methylprednisolone.

In a further preferred embodiment the calcineurin inhibitor is cyclosporin A, Voclosporin, and/or Tacrolimus.

In the following the invention is explained in detail by examples and figures. The figures show:

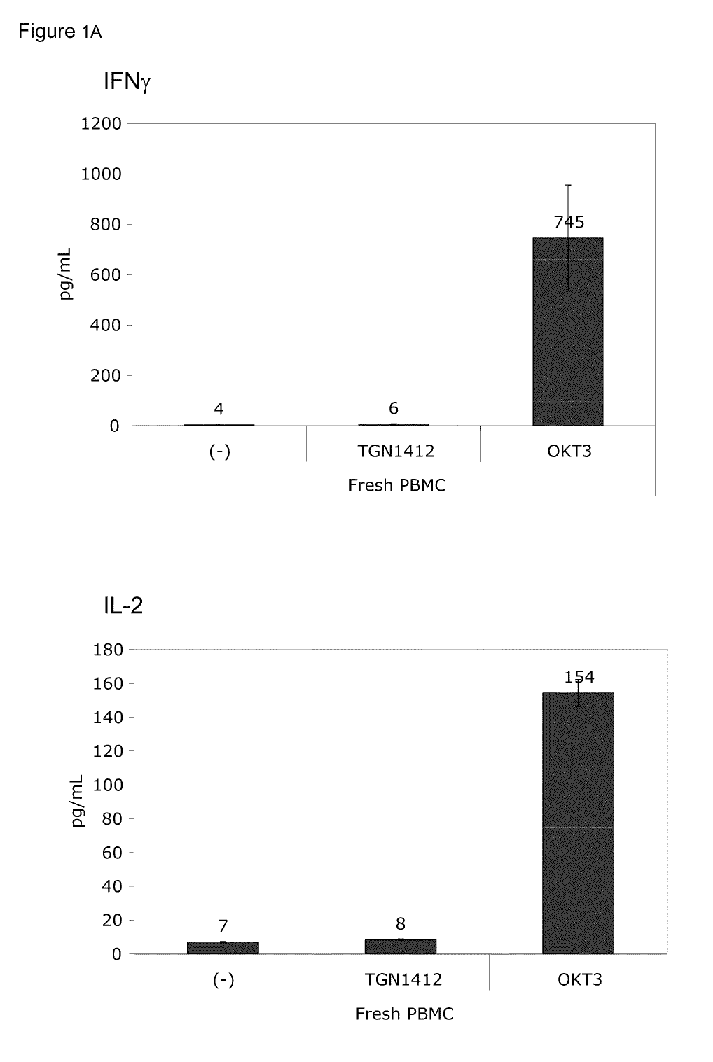

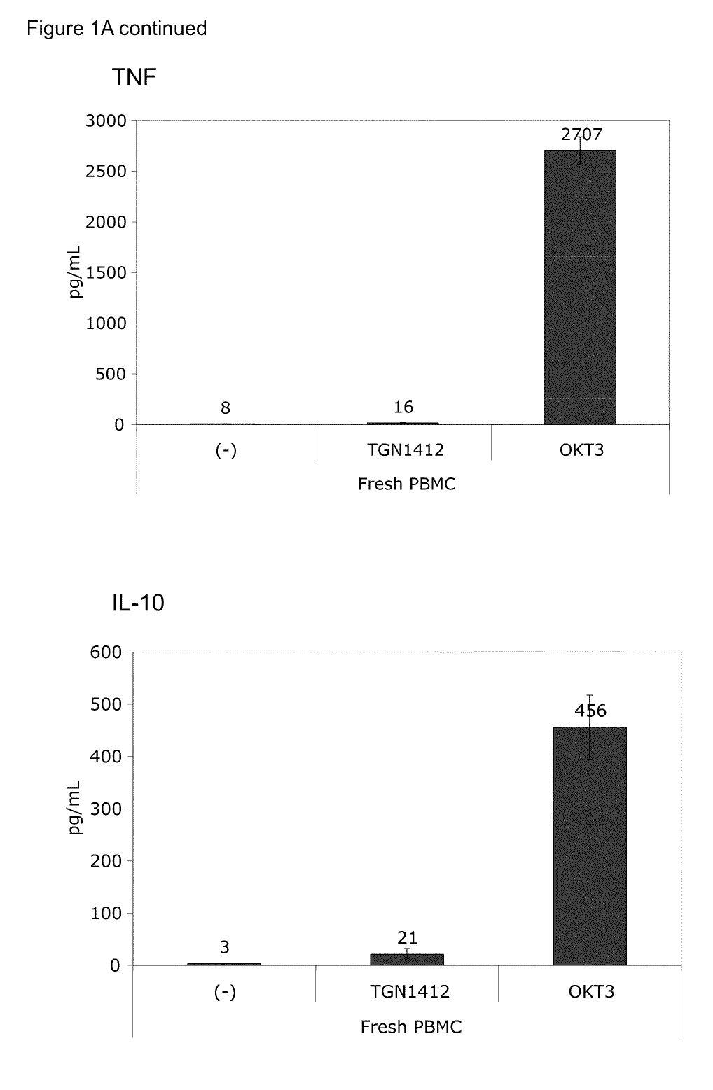

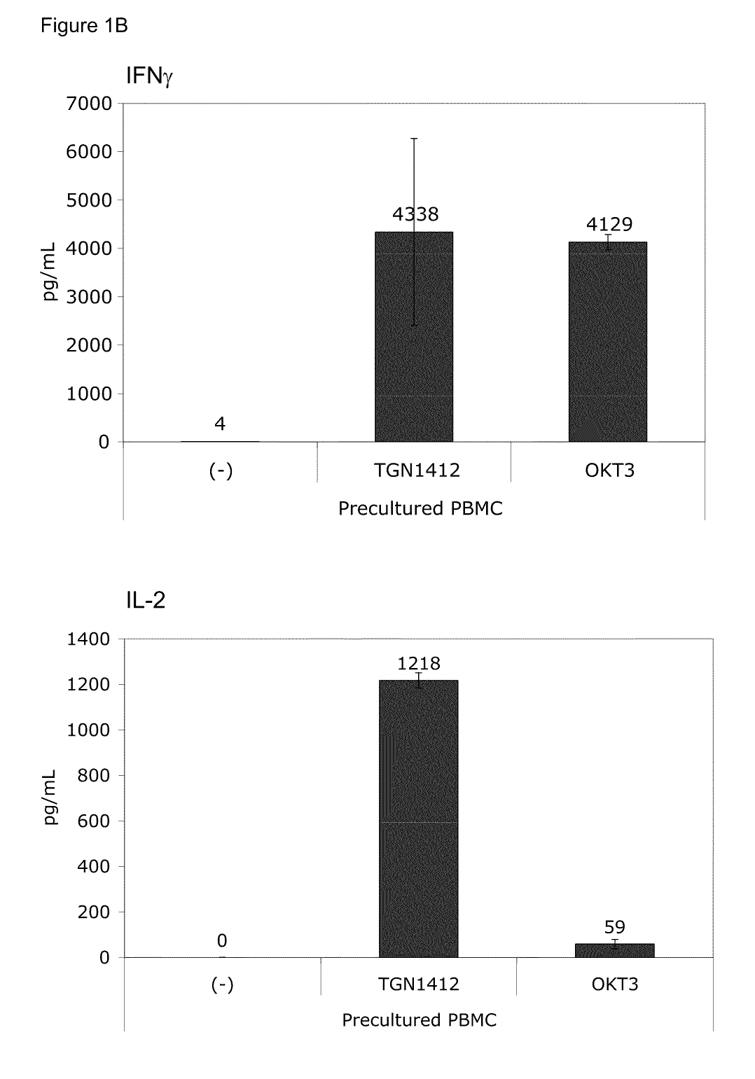

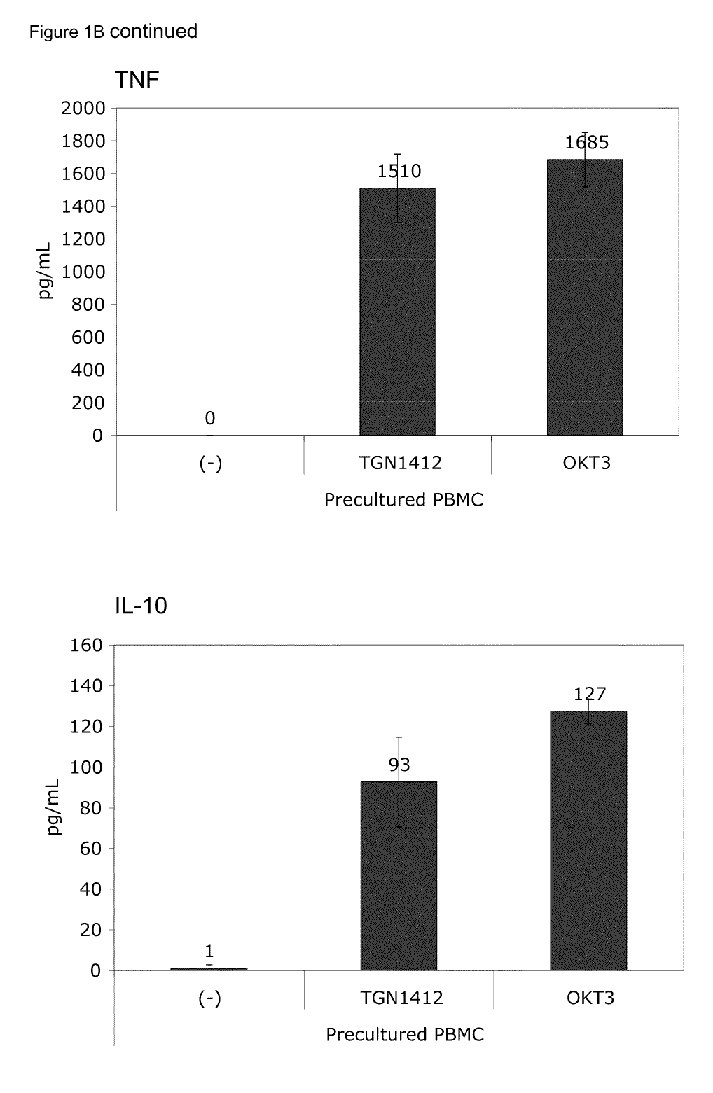

FIG. 1: Induction of cytokine release by the CD3-specific mAb OKT3 and the CD28 superagonist TGN1412 from human PBMCs.

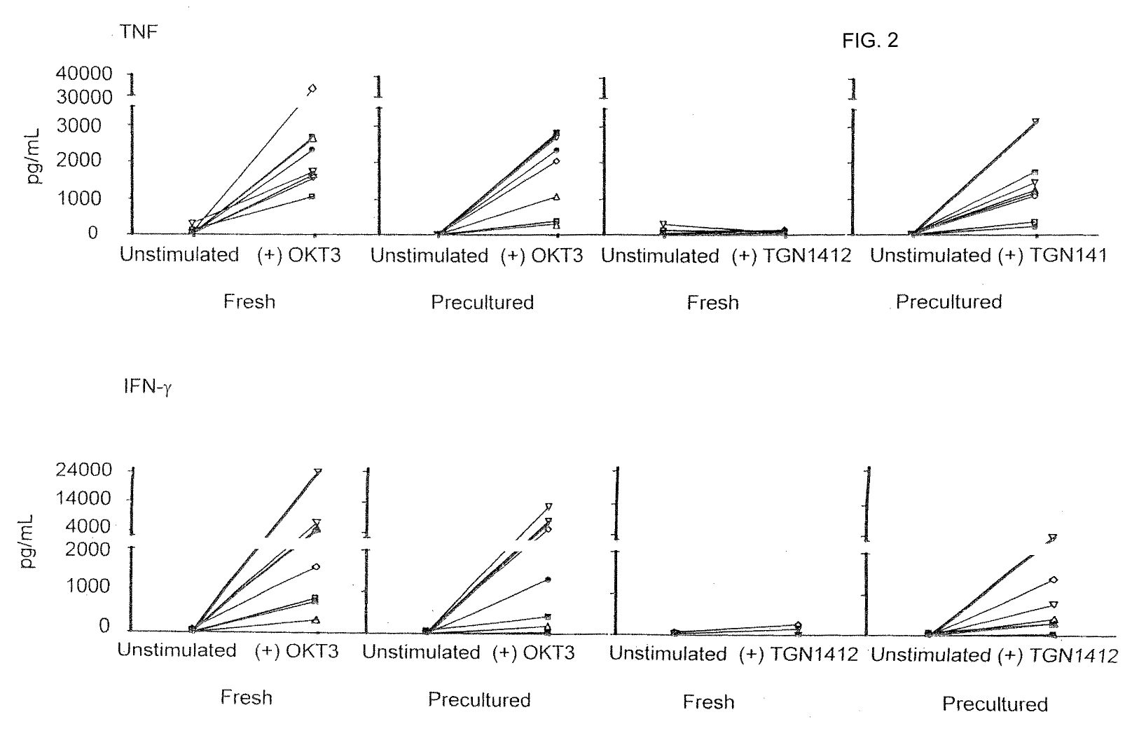

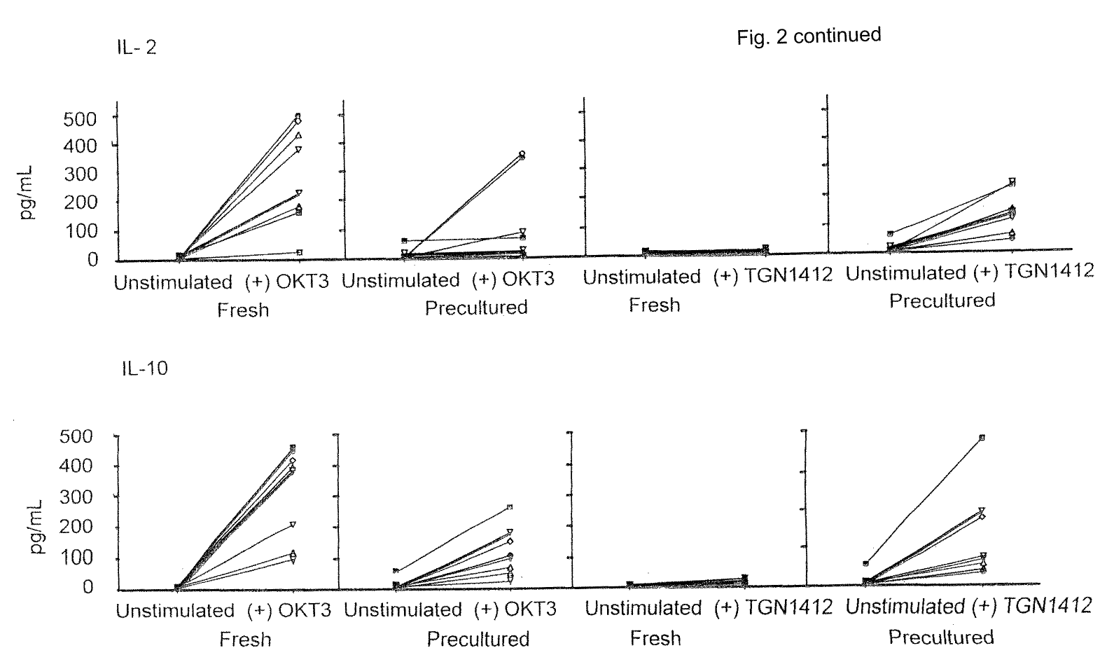

FIG. 2: Consistant conversion from a TGN1412 non-reactive to a TGN1412-reactive state by 2 day preculture.

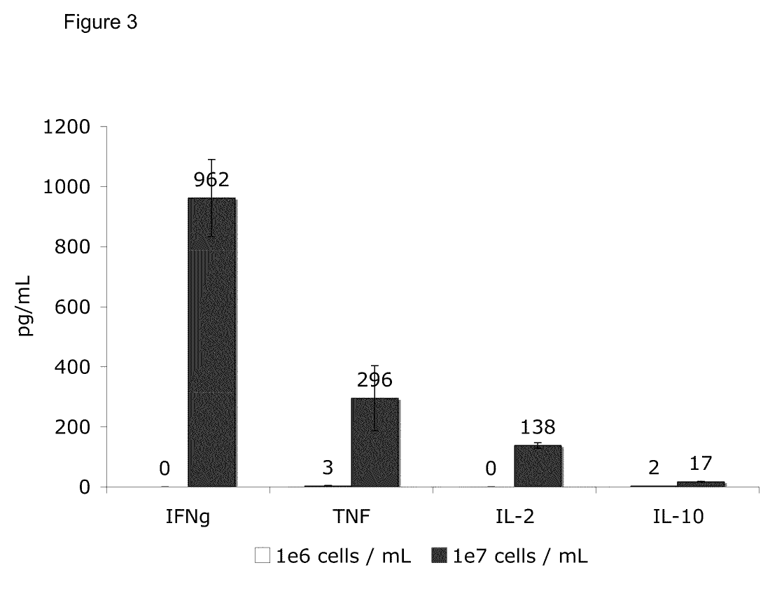

FIG. 3: Acquisition of TGN 1412-reactivity requires high cell density during preculture.

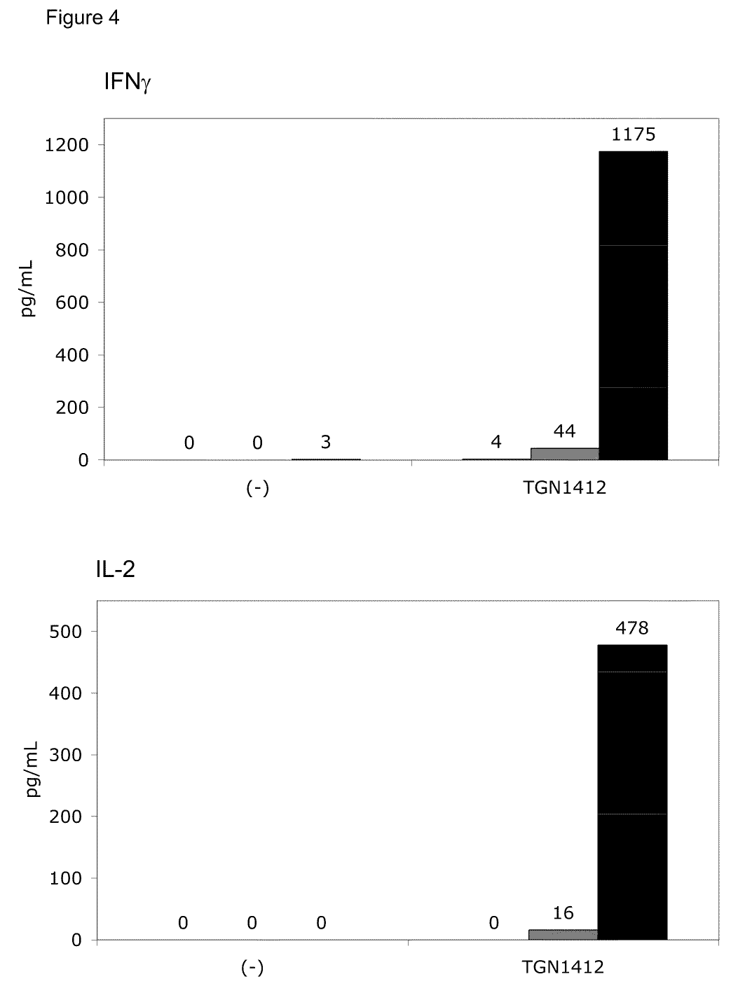

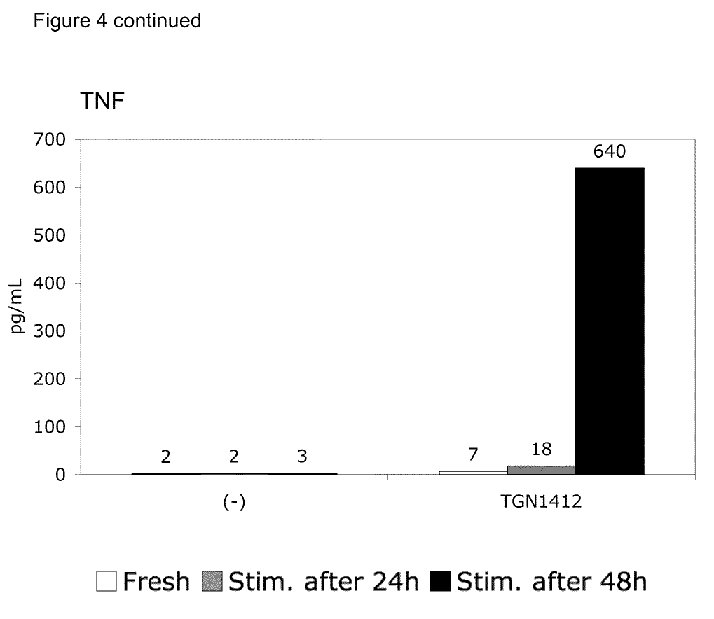

FIG. 4: Acquisition of TGN1412-reactivity in high-density precultures requires two days of incubation.

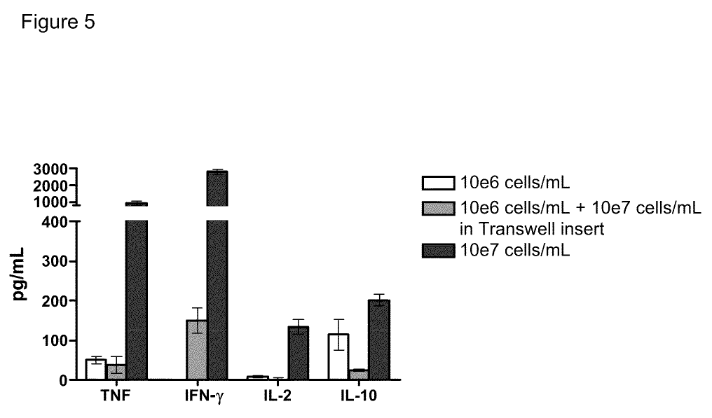

FIG. 5: Acquisition of TGN 1412-reactivity in high-density cultures requires cell-cell contact.

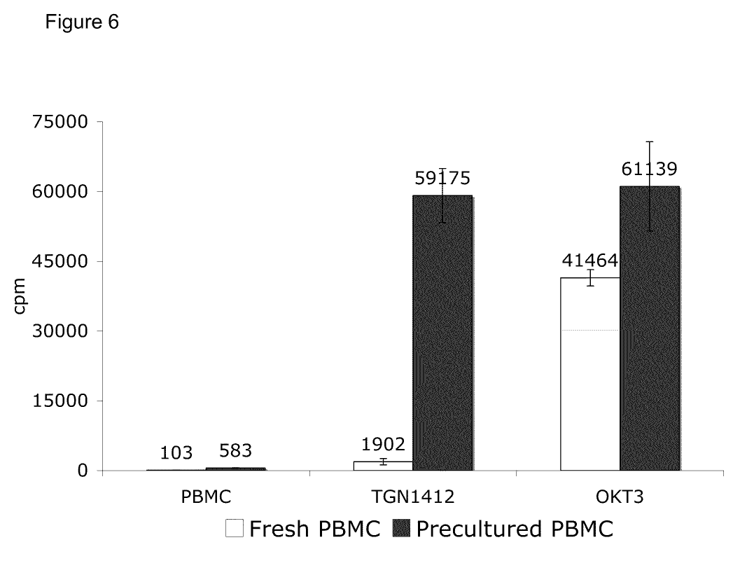

FIG. 6: Precultured, but not fresh PBMCs respond to TGN1412 with proliferation.

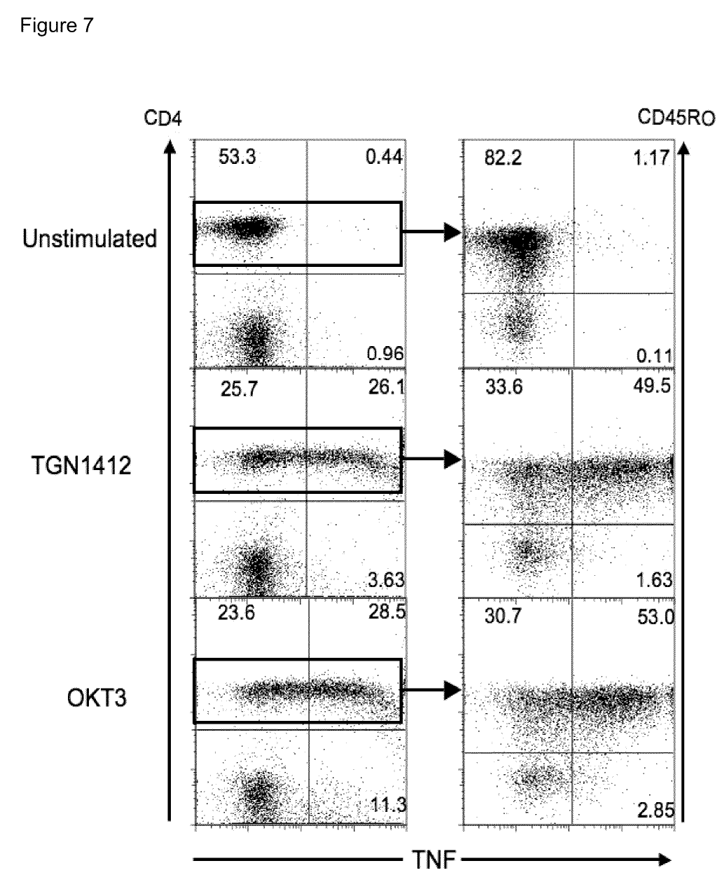

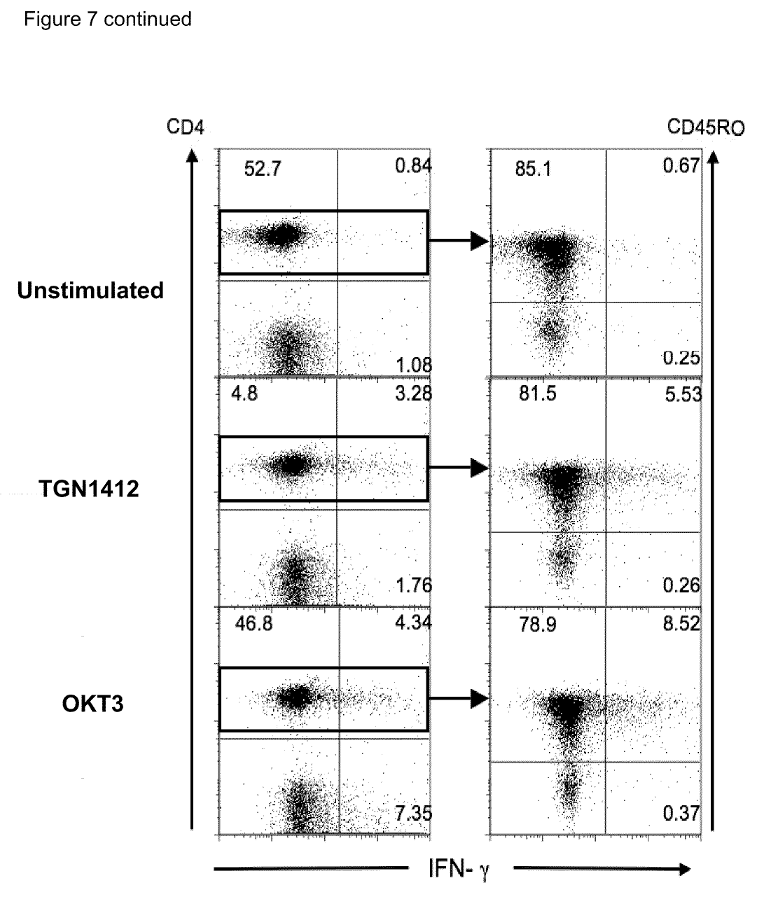

FIG. 7: CD45RO (memory) CD4 T-cells are the main source of proinflammatory cytokines released by both OKT3 and TGN1412.

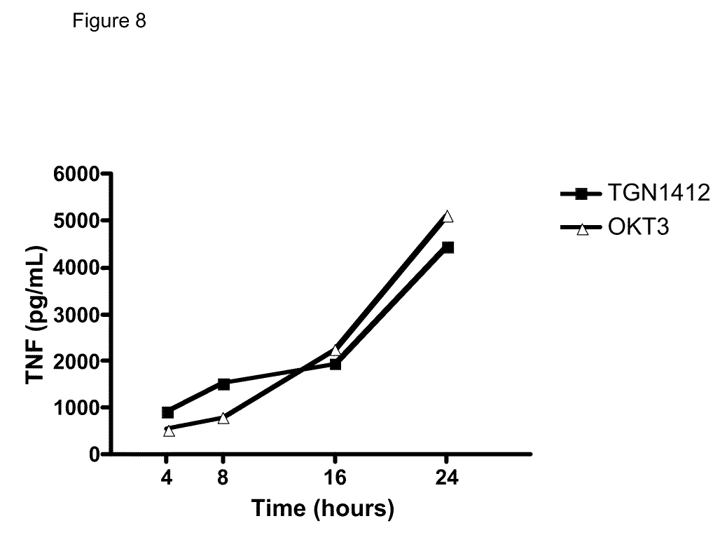

FIG. 8: Comparable kinetics of TNF release from high-density precultured PBMCs induced by OKT3 and TGN1412.

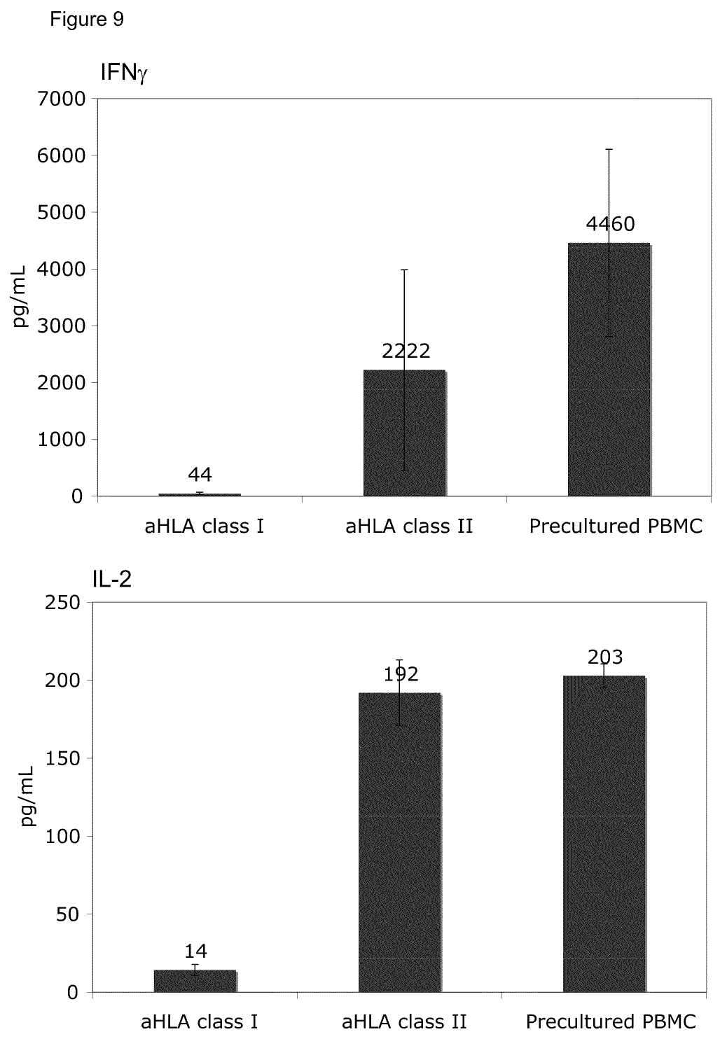

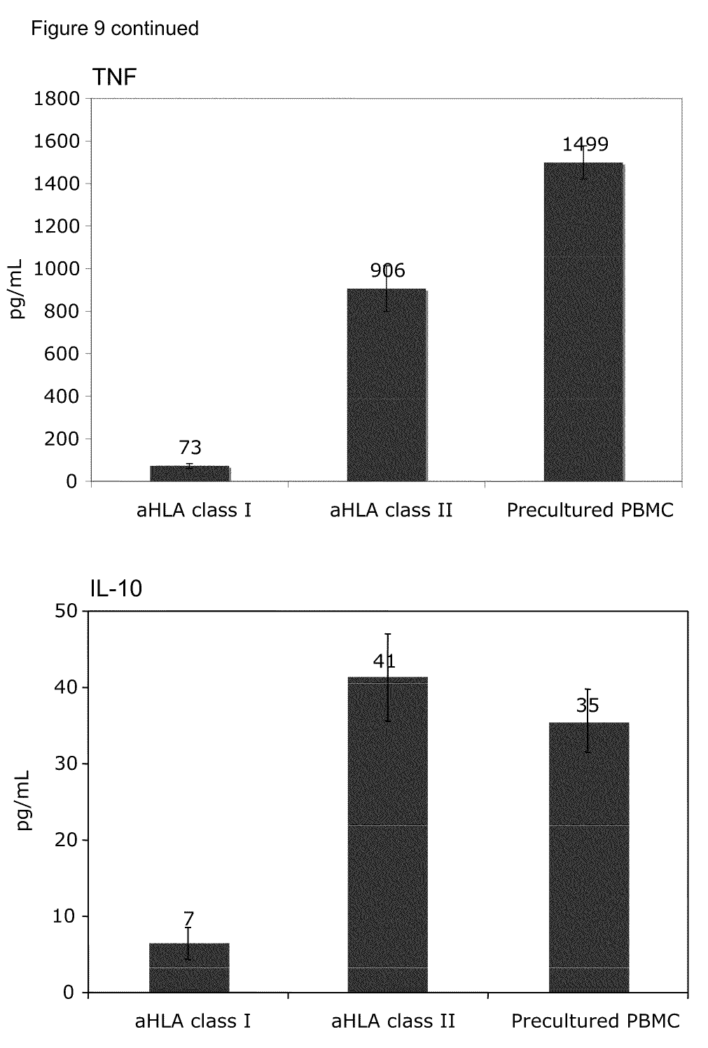

FIG. 9: Acquisition of TGN1412-reactivity is blocked by mAb to HLA antigens.

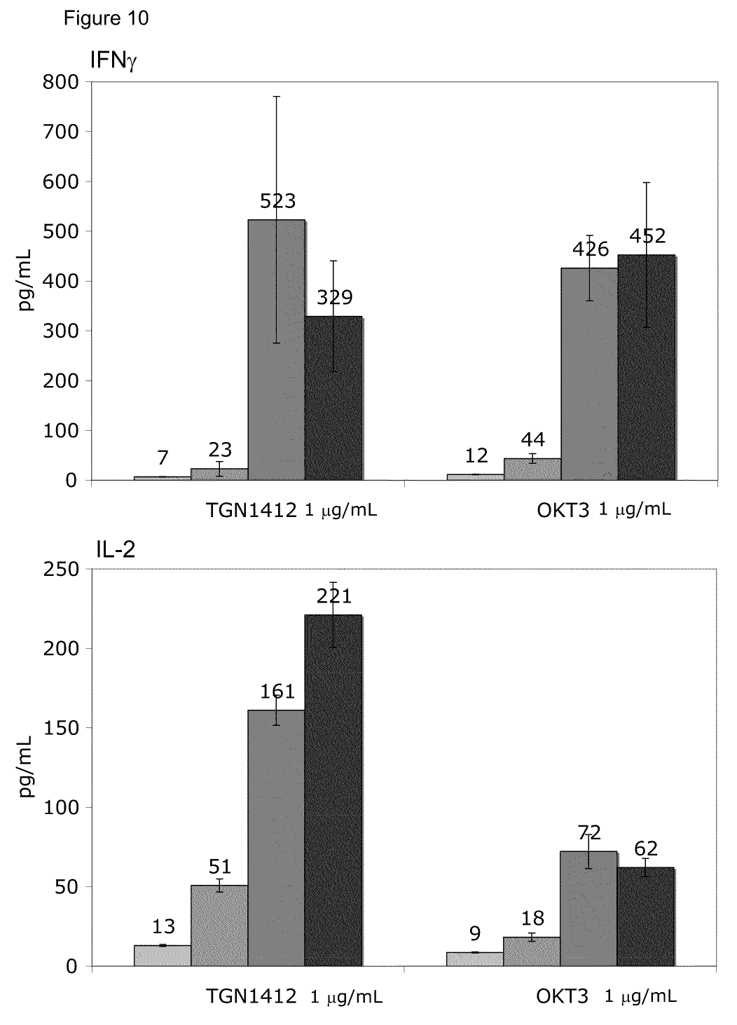

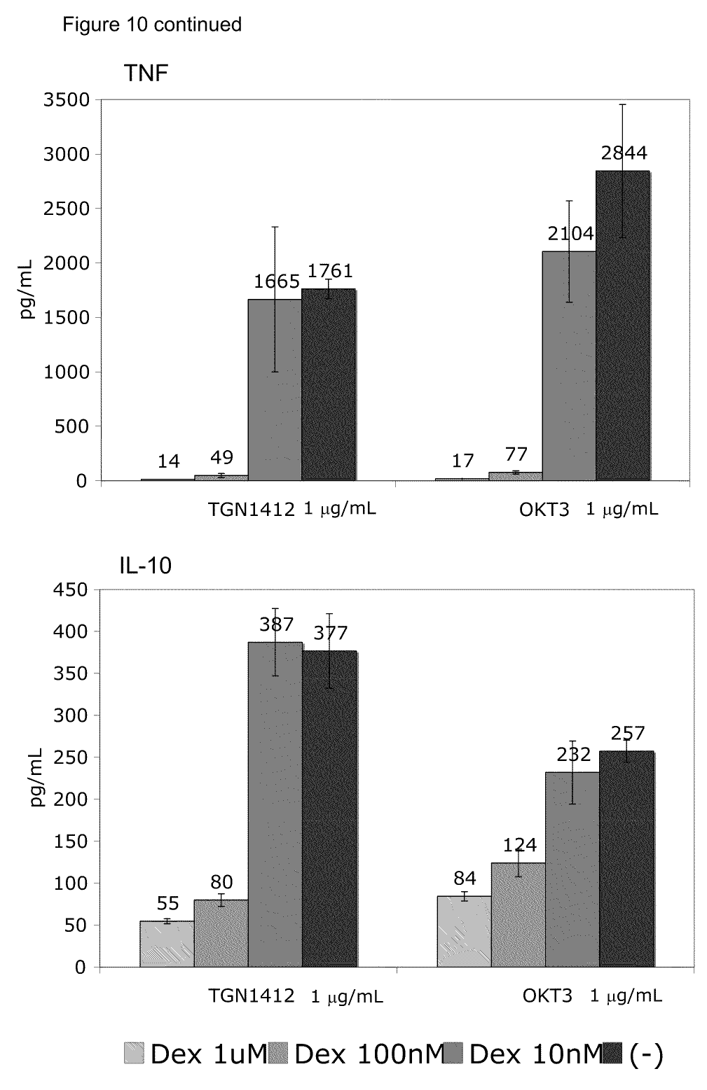

FIG. 10: Comparable sensitivity of cytokine release induced by OKT3 and TGN1412 to corticosteroid-mediated suppression.

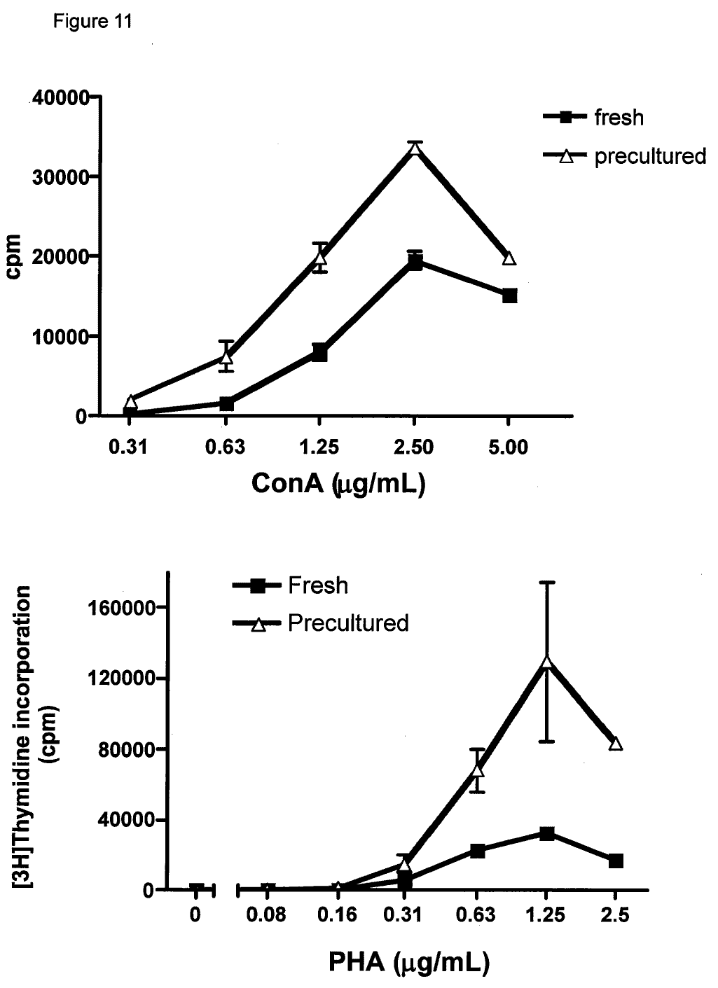

FIG. 11: Enhancement of proliferative T-cell response to ConA and PHA in precultured PBMCs.

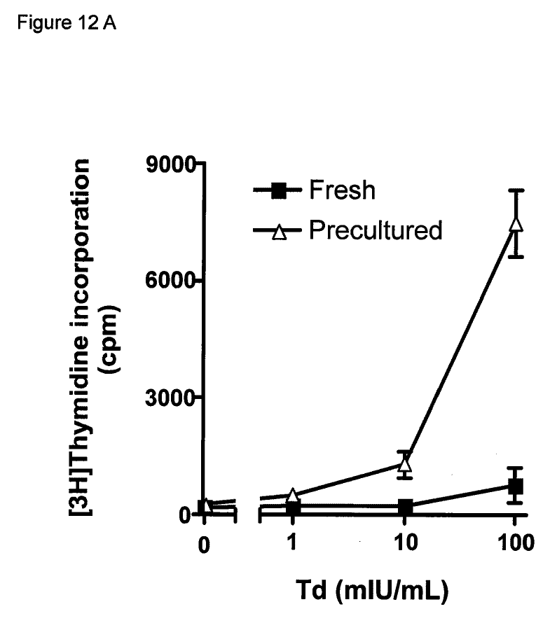

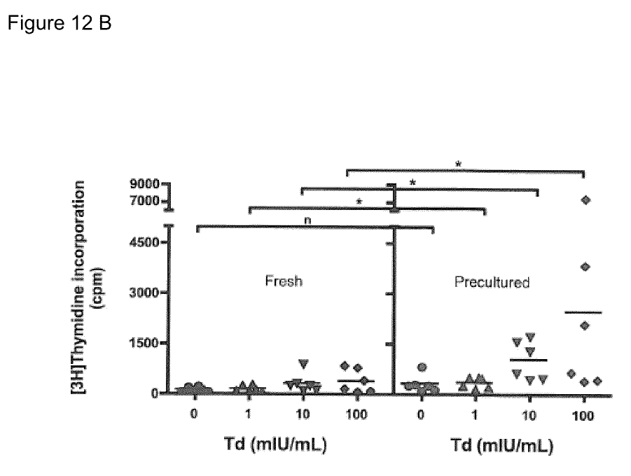

FIG. 12: Enhancement of recall response to tetanus/diphtheria toxoids in precultured PBMCs (A), compilation of data from six individual donors (B).

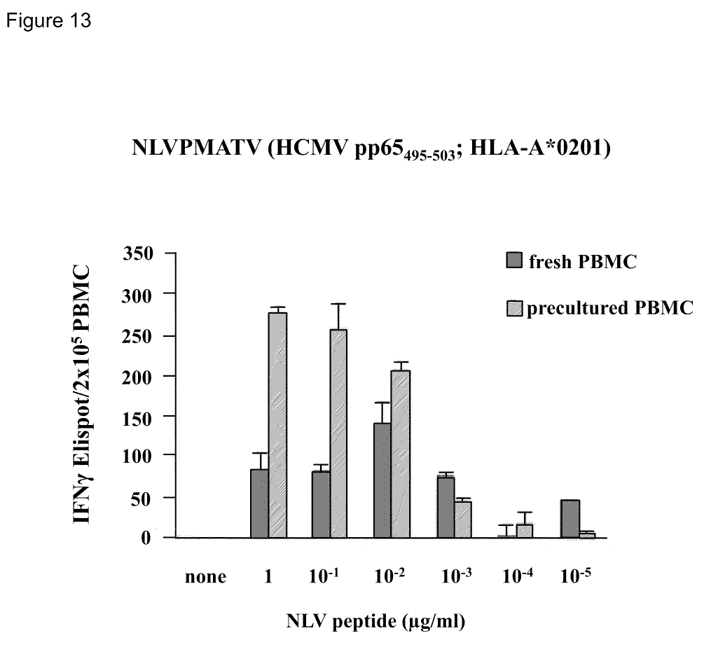

FIG. 13: Enhancement of proliferative T-cell response to a viral peptide in precultured PBMCs.

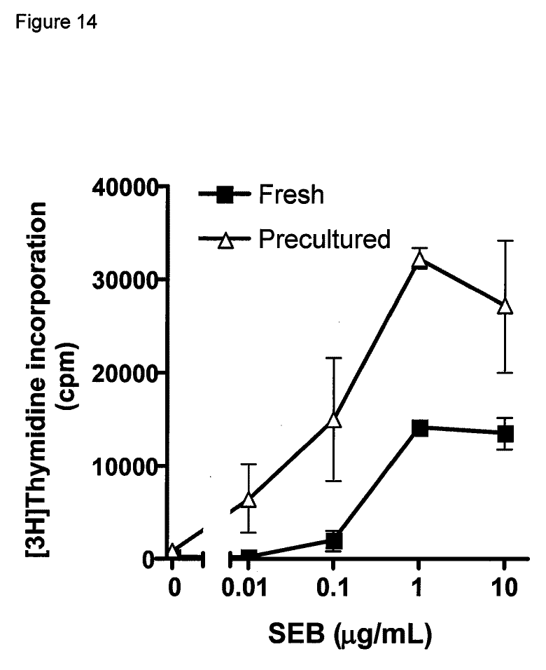

FIG. 14: Enhancement of proliferative T-cell response to staphylococcal enterotoxin B in precultured PBMCs.

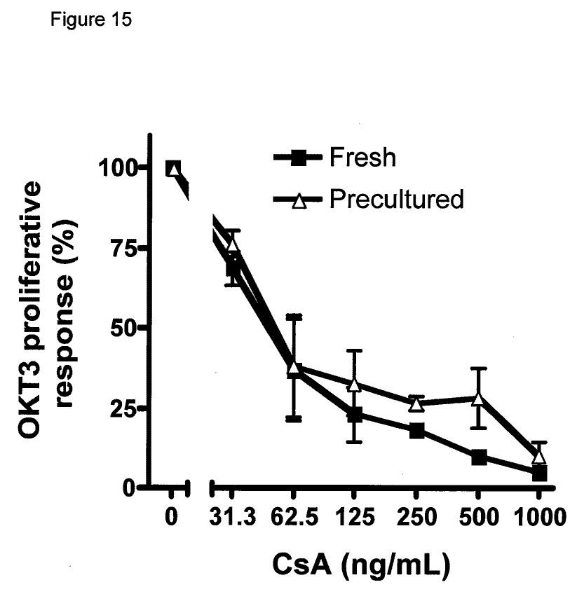

FIG. 15: Reduced suppression of proliferative T-cell response to high CsA doses in precultured PBMCs.

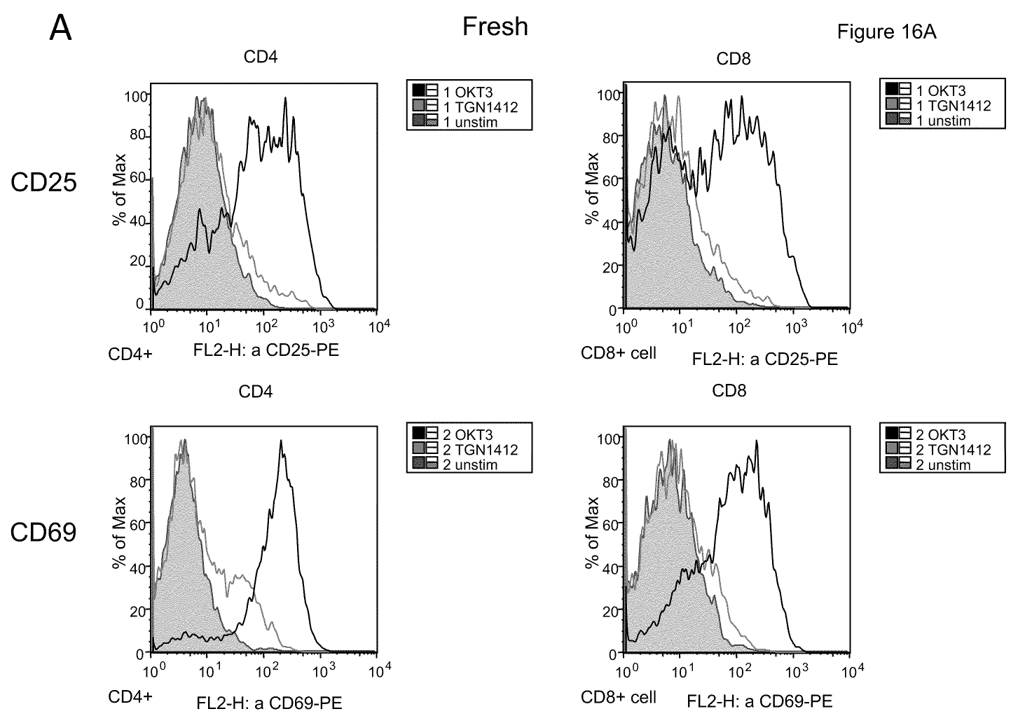

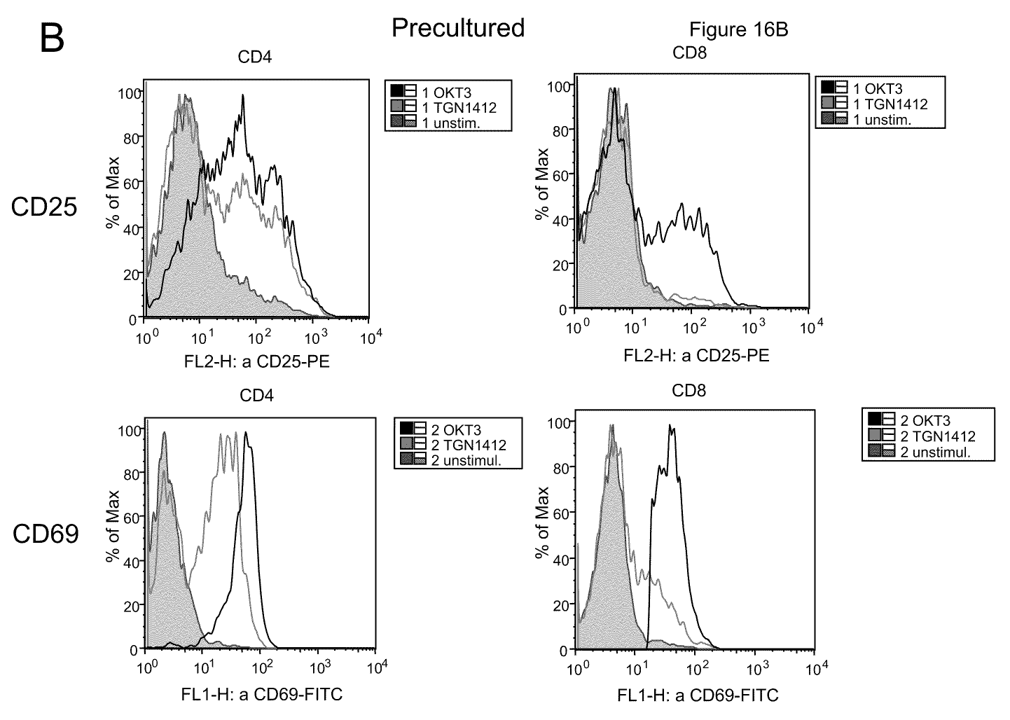

FIG. 16 Induction of activation markers on fresh (A) and precultured (B) T-cells by TGN1412 and OKT-3 stimulation. PBMC were used fresh (A) or were precultured under high density conditions for two days (B), harvested and restimulated under standard (low density) conditions with either 1 μg/ml OKT-3 (black line), 1 μg/ml TGN1412 (grey line), or without stimulating antibody (filled histogram). After 18 hrs., cells were harvested and stained with mAb specific for CD4, CD8, CD25, and CD69. Histograms shown are for CD25 (top) and CD69 (bottom) gated on CD4 (left) or CD8 T-cells (right).

The examples illustrate embodiments of the invention.

### EXAMPLE 1

### Comparative Example

For the induction of cytokine release, the present invention as well as this comparative example uses a system of PBMC stimulation as described below to study the response of human PBMCs to immune-modulating agents. This system employs freshly prepared PBMCs, isolated from heparinized venous blood by centrifugation over a density gradient (Lymphocyte Separation Medium LSM 1077, PAA Laboratories, Pasching, Germany) following the manufacturer's instructions. Alternatively, a fresh leukocyte concentrate, obtained from leukocyte reduction system chambers (Caridian, Gambro BCT, Lakewood, Colo., USA) as a byproduct in the preparation of platelet concentrates (Dietz et al., 2006) is used as starting material for Ficoll purification, with essentially the same results. PBMCs are cultured in 96-well tissue culture plates (Greiner bio-one, Frickenhausen, Germany), in which 2×10⁵ cells are stimulated in 0.2 ml of enriched RPMI 1640 culture medium (GIBCO/Invitrogen, Long Island, N.Y., USA) supplemented with 10% autologous serum or commercially available pooled AB serum (PAA Laboratories), with essentially the same results. When using commercially available pools of AB serum, these should be pretested in comparison to autologous serum in order to ensure that comparable results are obtained.

In this tissue culture system, freshly isolated PBMCs were stimulated with soluble TGN1412 provided by TheraMab GmbH, Wurzburg. It was of the same GMP-quality batch used during the London trial (Suntharalingam et al., 2006). As a positive control for the induction of cytokine release, clinical-grade OKT3 (“Muromonomab,” Janssen-Cilag, Neuss, Germany) was used, which is well known to trigger cytokine release both in vitro and in patients (Abramowicz et al., 1989). After 24 hours, a panel of cytokines including the major pro- and anti-inflammatory factors detected in plasma of the volunteers of the TGN1412 trial (Suntharalingam et al., 2006) were analyzed by the cytokine-bead-array (CBA) technology (Becton Dickinson, Mountain View, Calif., USA) following the manufacturer's instructions.

The concentration of both OKT3 and TGN1412 employed was 1 μg/ml, which is in the range of the estimated concentration achieved in the circulation of the volunteers during the London TGN1412 trial (Duff, 2006). Extensive titrations of both antibodies not displayed here showed that this concentration is within the optimum range for biological responses.

FIG. 1A shows that soluble TGN1412 fails to induce cytokine release in fresh PBMCs. In contrast, OKT3 is highly effective in inducing TNF, IFN-gamma, and IL-2, which are all known to contribute to the pathological manifestations of the cytokine release syndrome, as well as the anti-inflammatory cytokine IL-10. PBMCs from a healthy donor were isolated by Ficoll density centrifugation and cultured in 0.2 ml RPMI1640 medium supplemented with 10% AB serum for 24 hours at 10⁶/ml in 96 well flat bottom tissue culture plates. Cytokines in the supernatants were analyzed after 24 hours of incubation by cytokine bead array. Monoclonal antibodies were used at 1 μg/ml final concentration. Triplicate means and standard deviations are shown.

The failure of TGN1412 and the ability of OKT3 to induce release of these and other cytokines (not shown) in such standard PBMC cultures has been reproduced with over 10 individual donors, and is in agreement with the data submitted by TeGeneroAG and reproduced in the report of the Scientific Expert Group on the safety of Phase I clinical trials (Duff, 2006).

### EXAMPLE 2

### Response by TGN1412 After Preculturing

For the experiment in FIG. 1B PBMCs from a healthy donor were cultured in 1.5 ml medium for 2 days at 10⁷/ml in 24 well flat bottom tissue culture plates before being washed and readjusted to 10⁶/ml. With these cells, the same experiment was performed as described in Example 1.

FIG. 1B shows that surprisingly, responsiveness to TGN1412 is restored by simply preculturing PBMCs for 2 days without overt stimulation. When cells were prepared on Dec. 4, 2008, the number of PBMCs obtained exceeded that required for the current experiment, and surplus cells were stored in culture medium for two days at 37° C. When these cells were employed for exactly the same experiment as previously performed with fresh cells (FIG. 1A), something completely unexpected happened: TGN1412 now induced a cytokine release of comparable magnitude as OKT3. FIG. 1B provides an example of such an experiment.

The reproducibility and mechanistic basis were therefore further investigated.

### EXAMPLE 3

### Reproducibility of the Key Observation

FIG. 2 summarizes the effect of preculture on the reactivity to TGN1412 for 7 individual healthy donors. Data from 7 individual healthy donors are shown, each represented by a symbol. Conditions for antibody stimulation and for preculture were as in FIG. 1. While donor-specific variations exist for both, OKT3 and TGN1412 responses, it is apparent that in all cases, fresh donor cells failed to respond to TGN1412 stimulation with cytokine release and that this refractory state was lost after 2 days of preculture. Donor-specific variations are expected, as is illustrated by the great differences in the magnitude of the cytokine storm experienced by the volunteers of the London TGN1412 trial (Suntharalingam et al., 2006).

### EXAMPLE 4

### Optimization of the New Method

FIGS. 3 to 5 describe the parameters determining the acquisition of sensitivity to TGN1412 in peripheral blood T-cells.

For the experiment of FIG. 3 PBMCs were cultured at 10⁶/ml (left bars) or at 10⁷/ml (right bars) for 2 days before being stimulated by TGN1412 at 10⁶/ml as described in FIG. 1.

For the experiment of FIG. 4 fresh PBMCs (left bars of triplets), and PBMCs precultured at 10⁷/ml for 24 (middle bars of triplets) or 48 hours (right bars of triplets) were stimulated with OKT3 or TGN1412 for 24 hours under the conditions given in FIG. 1.

For the experiment of FIG. 5 PBMCs were cultured for two days at high density (10⁷/ml, right bars of triplets) or at low density (10⁶/ml, left and middle bars of triplets) in 1.5 ml cultures. In the group shown as middle bars of triplets, these low density cultures additionally contained an insert with a semipermeable membrane on which additional PBMCs were cultured at high-density (10⁶/ml below membrane, 10⁷/ml above membrane, middle bars of triplets). Cells were restimulated and assayed as given in FIG. 1.

As shown above, PBMCs acquire sensitivity to TGN1412 by preculture in medium with 10% autologous or commercially available AB serum. We tested the role of the following parameters in the acquisition of TGN1412 sensitivity.

Cell density. In contrast to standard PBMC stimulation assays where cells are cultured at 10⁶/ml or 2×10⁵/cm² of the culture well, “parking” for 2 days was performed at about a 10 fold higher density. FIG. 3 shows that preculturing PBMCs at high (10⁷/ml or 2×10⁶/cm²), but not at low (10⁶/ml or 2×10⁵/cm²) density induces reactivity to TGN1412 in the secondary culture. Because of the requirement of cell density and culture for two days, one half of the culture medium is carefully replaced with fresh pre-warmed medium after 24 hours in order to supply the cells with sufficient nutrients.

Time. FIG. 4 shows that full reactivity to TGN1412 (comparable to reactivity to OKT3) is achieved after 2 days of pre-culture. 1 day of preculture leads only to a modest increase of reactivity.

Requirement for cell-cell contact. The requirement for high cell density during preculture of PBMCs for the acquisition of reactivity to TGN1412 can be due to the need for cell-cell contact and/or to the action of soluble factors which need to reach a certain concentration to promote maturation to the reactive state. Using a transwell system (Corning incorporated, Lowell, Mass., USA), where cells cultured at high density are separated from those cultured at low density by an 8 μm-pore membrane permitting the diffusion of soluble factors, it is shown in FIG. 5 that cell-cell contact is required.

### EXAMPLE 5

### Further Characteristics of the New Method

FIG. 6 shows that precultured, but not freshly isolated PBMCs proliferate in response to TGN1412. Fresh and precultured PBMCs were prepared and cultured as described for FIGS. 1 A and B, respectively. On day 3, 1 μCi of 3H-thymidine was added, and cultures were harvested 16 hours later and processed for liquid scintillation counting. Besides cytokine release, polyclonal T-cell activation results in proliferation which can be measured as radioactivity incorporated, here incorporated as tritiated thymidine. As is seen in FIG. 6, OKT3 stimulated the proliferation of both, fresh and precultured PBMCs, whereas TGN1412 was only able to induce proliferation in precultured PBMCs. Thus, proliferation can also be used as a readout.

FIG. 7 shows that in precultured PBMCs, TGN1412 releases proinflammatory cytokines from CD4 memory cells. 2 day high-density precultured PBMCs were stimulated for 18 hours with 1 μg/ml of OKT3 or of TGN1412. Instead of 18 hours stimulation for a period of time between 15 to 20 hours, preferably 16 to 18 hours, is generally possible. During the last 4 hours of culture, 5 μg/ml of brefeldin A was added to block cellular export of cytokines. After cell-surface staining with fluorochome-conjugated mAb to CD4 and CD45RO (memory marker), cells were fixed, permeabilized and stained with mAb to TNF—left panels—or IFNgamma—right panels—(all from BD Pharmingen, Mountain View, Calif., USA)

15.000 live-gated events were acquired on a BD Calibur flow cytometer and data were analyzed using FlowJo software (Three Star Inc., Ashland, Oreg., USA). The cytokine storm elicited by OKT3 in patients is a well-known phenomenon, and the accompanying information provided with this drug (Muromonomab, Janssen-CILAG) explicitly warns against this syndrome. To compare the cellular source of the two key pro-inflammatory cytokines TNF and IFN-gamma in response to OKT3 and TGN1412, they were retained within the cell by blockade of their transport through the Golgi apparatus using the drug brefeldin A (Sigma Aldrich, Steinheim, Germany), and revealed by intracellular staining of fixed and permeabilized cells with fluorescent TNF- and IFNgamma-specific mAbs, respectively (Becton Dickinson, Mountain View, Calif., USA). In parallel, the cell surface phenotype of the stimulated PBMCs was determined. The main cytokine-producing T-cell subset, the CD4 T-cells, were thus identified and further subdivided into naive (CD45R0−) and memory (CD45RO+) subsets. FIG. 7 shows that in both fresh and precultured PBMCs, OKT3 and TGN1412 trigger cytokine production predominantly in the CD4 memory subset.

FIG. 8 shows that TNF release from precultured PBMCs follows the same kinetics when induced by either OKT3 or TGN1412. High-density precultured PBMCs were prepared as in FIG. 1B and stimulated with 1 μg/ml of OKT3 or TGN1412. Supernatants were harvested at the times indicated and TNF content was analysed as in FIG. 1.

In vivo, the release of TNF, the most important pro-inflammatory cytokine of the “cytokine storm”, follows the same kinetics when induced by either OKT3 or TGN1412 (Abramowicz et al., 1989; Suntharalingam et al., 2006). Therefore the kinetics of TNF-release in precultured PBMCs between the two mAb were compared and found to be virtually the same.

### EXAMPLE 6

### Mechanistic Basis for the Induction of Reactivity to TGN142 During High-Density Preculture: Self-MHC Recognition

This Example provides functional information about the invention without being bound to theory. It has been suggested from work done in mice that recognition of molecules of the major histocompatibility complex (MHC; HLA in humans) by the antigen receptors (TCR) of T-cells in the lymphoid organs (lymph nodes, spleen, etc.) primes the TCR for enhanced signalling during antigen encounter later on (Stefanova et al., 2002). This process is referred to as “MHC-scanning” and describes the constant interaction of the TCR of tissue-resident T-cells with HLA molecules on their neighbouring cells irrespective of the peptides they contain (Stefanova et al. 2002). This interaction leads to the priming of the TCR and the assembly of a signaling platform which facilitates full T-cell activation when a fitting antigenic peptide is encountered by the TCR. This process is also referred to in the art as “tonic” TCR signaling. Blocking mAbs which react with all human HLA class I and class II molecules were included during the two days of preculture (mAbs 646-2.6 and Tü39, Becton-Dickenson) at 10 μg/ml.

FIG. 9 shows that HLA class I, and to a lesser extent HLA class II specific mAb are able to block acquisition of TGN1412 reactivity, illustrating the need for HLA recognition by the TCR of the cells acquiring this reactivity. High-density preculture of PBMCs was performed as given in FIG. 1B. To some high-density precultures, mAb to HLA class I or HLA class II were added at 10 μg/ml.

Interaction of the TCR with HLA molecules in the densely packed lymphoid organs is a prerequisite for the strong reactivity to TGN1412 as experienced by the volunteers in the London trial, and that this situation is mimicked by high-density culture in vitro, thus restoring the reactivity of circulating T-cells, which have lost cell-cell contact, to that of the T-cells in lymphoid organs. It also explains why the response to OKT3 is observed not only in precultured but also in fresh PBMCs. Since in contrast to TGN1412, OKT3 addresses the TCR itself, there is no need for “priming” this receptor by interactions with HLA-molecules. The differential ability of HLA class I and - class II specific mAb to block the acquisition of reactivity to TGN1412 is explained by the fact that more than 90% of available HLA molecules in PBMC cultures are of class I.

### EXAMPLE 7

### Testing the Ability of Corticosteroids to Control TGN1412-Mediated Cytokine Release

The OKT3-induced cytokine storm is routinely managed by prevention or intervention with high-dose corticosteroids (Goldman et al., 1989). Until now, it was impossible to test the sensitivity of TGN1412-induced cytokine release to corticosteroids because no assay system existed. We therefore used the new method to compare the sensitivity of cytokine release in fresh and precultured PBMCs to dexamethasone (“Dex”, Sigma-Aldrich).

FIG. 10 shows that TGN1412-induced cytokine release is sensitive to corticosteroids. High-density precultured cells were prepared and stimulated by mAb as described in FIG. 1B. Where indicated, dexamethasone was included at the final concentrations given, and cytokines were measured after 24 hours of culture.

Cytokine release induced by both, OKT3 and TGN1412 is fully suppressed by the highest dose employed (1 μM), and is still almost completely suppressed at a tenfold lower dose (100 nM). This strongly suggests that TGN1412 induced cytokine release can be controlled by appropriate corticosteroid medication as is clinically used for OKT3-treated patients.

### EXAMPLE 8

### Reactivity to Mitogenic Lectins

The mitogenic lectins concanavalin A (ConA) and phytohemagglutinin (PHA) are routinely used to test the T-cell reactivity in PBMC preparations. These lectins act through one or several cell surface glycoproteins where they bind to sugar moieties. FIG. 11 shows that for both lectins, the proliferative T-cell response is strongly enhanced if PBMCs were precultured for two days following the above protocol.

### EXAMPLE 9

### Antigen-Specific Recall Response

As a result of intentional vaccination or natural infection, the frequency of T-lymphocytes specific for a given microbial antigen increases to a level where restimulation in vitro allows the detection of proliferative responses. Classical examples for such antigens are diphteria and tetanus toxoids. Such preparations are routinely used to assess the vaccination status of individuals. For this purpose, the two toxoids are offered as a single preparation by Sanofi Pasteur. FIG. 12A illustrates that indeed, the recall response to tetanus/diphteria toxoids is strongly enhanced by preculturing PBMCs at high density for two days. In FIG. 12B, data from six individual donors are compiled. * indicates statistical significance p<0.05

### EXAMPLE 10

### Response of CD8 T-cells to a Cytomegalo Virus Derived Peptide

Recall responses to tetanus and diphteria toxoid address CD4 T-cells. To test whether the recall response of CD8 T-cells is also affected by preculture at high cell density, PBMCs of an HLA A2 positive individual previously exposed to cytomegalo virus were stimulated with a peptide presented by HLA A2 to the individual's CD8 T-lymphocytes. As a read-out, the number of IFNgamma secreting CD8 T-cells was chosen because it is known that such CD8 memory T-cells rapidly secrete IFNgamma when stimulated through the antigen receptor. The quantification was performed using a so-called elispot assay, where PBMCs are cultured on a filter membrane coated with an IFNgamma reactive monoclonal antibody. The IFNgamma thus captured during a 24 h incubation period is then visualized using a second, enzyme tagged mAb and the conversion of a colourless to a coloured substrate. As shown in FIG. 13, preculture of PBMCs for two days at high cell density strongly enhanced the frequency of IFNgamma-producing cells in response to the viral peptide.

### EXAMPLE 11

### Response to the “Superantigen” Staphylococcal Enterotoxin B

Some bacteria secrete superantigens which are able to stimulate a much larger fraction of T-lymphocytes (5-20%) through their antigen receptors than is the case for conventional antigens (1/100,000 in a non-immunized individual). Accordingly, a proliferative response to such superantigens can be measured in vitro using PBMCs. FIG. 14 shows that again, preculture of PBMCs at high density for two days strongly enhances the sensitivity of this assay.

### EXAMPLE 12

### Testing Immunosuppressant Drugs

Potency of immunomodulatory drugs can be misjudged when using fresh rather than precultured PBMCs. Cyclosporin A (CsA) is a widely used immunosuppressant drug, which acts in the signal transduction pathway of the T-cell antigen-receptor. Accordingly, the reduced signaling capacity of circulating T-cells could translate into increased sensitivity to this drug. FIG. 15 shows that this is indeed the case: while both fresh and precultured PBMCs (from the same original preparation) respond to stimulation of the TCR with OKT3, precultured PBMCs are not as easily suppressed by high CsA doses as fresh PBMCs (p<0.03).

### EXAMPLE 13

### Induction T Cell Activation Markers on the Cell Surface

FIG. 16 shows induction of activation markers on fresh (A) and precultured (B) T-cells by TGN1412 and OKT-3 stimulation. PBMC were used fresh (A) or were precultured under high density conditions for two days (B), harvested and restimulated under standard (low density) conditions with either 1 μg/ml OKT-3 (black line), 1 μg/ml TGN1412 (grey line), or without stimulating antibody (filled histogram). After 18 hrs., cells were harvested and stained with mAb specific for CD4, CD8, CD25, and CD69. Histograms shown are for CD25 (top) and CD69 (bottom) gated on CD4 (left) or CD8 T-cells (right).
Abramowicz, D. et al., Transplantation 47, 606-608 (1989).
Dennehy, K. M. et al., J Immunol 178, 1363-1371 (2007).
Dietz, A. B. et al., Transfusion 46, 2083-2089 (2006).
Duff, G. W. C. Expert Scientific Group on Phase One Clinical Trials Final Report (Norwich, UK, Stationary Office 2006).
Gogishvili, T. et al., PLoS ONE 4, e4643 (2009).
Goldman, M. et al., Lancet 2, 802-803 (1989).
Hunig, T., Adv Immunol 95, 111-148 (2007).
Nguyen, D. H. et al., Proc Natl Acad Sci USA 103, 7765-7770 (2006).
Schraven, B. et al., Immunity 28, 591-595 (2008).
Stefanova, I. et al., Nature 420, 429-434 (2002).
Suntharalingam, G. et al., N Engl J Med 355, 1018-1028 (2006).

1. A method for testing a prospective or known immunomodulatory drug for T-cell activation, comprising the step of contacting in-vitro a peripheral blood mononuclear cell (PBMC) culture with a predetermined amount of the prospective or known immunomodulatory drug and observing the PBMC culture for T-cell activation using a readout system, upon contact with the prospective or known immunomodulatory drug, wherein the cell density of a PBMC preculture is adjusted such that cell-cell contact of the PBMC is enabled and wherein the PBMC preculture is cultured for at least 12 h.

2. The method according to claim 1, wherein the readout system observes the PBMC culture for release of at least one cytokine from the PBMCs, observes cell proliferation or is another suitable readout system, like changes in gene expression, protein expression and/or posttranslational modifications.

3. The method according to claim 1, wherein the preculturing step is carried out by storing the PBMC culture for at least 24 h, preferably at least 36 h, more preferably at least 45 h, at 35° C. to 40° C., preferably at 36° C. to 38° C., in absence of immunomodulatory drugs, and prior to the contact with the immunomodulatory drug to be tested.

4. The method according to claim 1, wherein the cell density of the PBMC culture during the preculture step is at least 2*10⁶/ml, preferably at least 5*10⁶/ml, more preferably at least 10⁷/ml, or at least 4*10⁵/cm², preferably at least 10⁶/cm², most preferably at least 2*10⁶/cm².

5. The method according to claim 1, wherein the immunomodulatory drug is an immunostimulating drug, like an antibody, preferably a monoclonal antibody, in particular a human CD28 specific superagonistic monoclonal antibody, or selected from the group consisting of lectines, like concanavalin A (ConA), or phytohemagglutinine (PHA), natural extracts comprising lectins, like echinacea extract, or mistletoe extract, superantigens, like Staphylococcal Enterotoxin B (SEB), Staphylococcal Enterotoxin A (SEA), Toxic Shock Syndrome Toxin-1 (TSST-1), staphylococcal pyrogens, like Staphylococcal Pyrogene Enterotoxin B (SPEB), superantigens produced by mycoplasma, like Mycoplasma arthriditis, or by black plague bacteria, like Yersinia pseudotuberculosis, superantigens produced by certain pathogenic viruses, like EBV or HIV-1.

6. The method according to claim 2, wherein the cytokine observed is selected from the group consisting of TNF, IFN-gamma, IL-1-beta, IL-1, IL-2, IL-3, IL-4, IL-5, IL-6, IL-7, IL-8, IL-9, IL-10, IL12p70, IL-13, IL-14, IL-15, IL-16, IL-17, IL-21, IL-35, and LT, and combinations thereof.

7. The method according to claim 1, wherein the precultured PBMC culture is additionally contacted with a prospective drug for attenuating the release of at least one cytokine, at the same time as contacting of the PBMC culture with the known immunomodulatory, in particular stimulating, drug or subsequently after a predetermined period of time, or a predetermined period of time there before, wherein the cytokine release is further observed.

8. The method according to claim 7, wherein the predetermined period of time is in the range of 10 s to 12 h, preferably in the range of 10 s to 1 h.

9. The method according to claim 7, wherein the prospective drug for attenuating the cytokine release is a corticosteroide.

10. The method according to claim 7, wherein the prospective drug for attenuating the cytokine release is an immunosuppressive drug selected from the group consisting of Rapamycin or calcineurin inhibitors, like cyclosporine A, Voclosporin, or Tacrolimus.

11. A corticosteroid, Rapamycin and/or a calcineurin inhibitor for use in treating, attenuating or preventing a cytokine storm upon administration of an anti-human CD28 specific superagonistic monoclonal antibody.

12. The corticosteroid and/or a calcineurin inhibitor for use of claim 11, wherein the antibody is TGN1412.

13. The corticosteroid and/or a calcineurin inhibitor for use of claim 11, wherein the corticosteroid is dexamethasone and/or methylprednisolone.

14. The corticosteroid and/or a calcineurin inhibitor for use of claim 11, wherein the calcineurin inhibitor is cyclosporin A, Voclosporin, and/or Tacrolimus.
